(19) United States
(12) Patent Application Publication
(10) Pub. No.: US 2022/0346335 A1
(43) Pub. Date: Nov. 3, 2022

(54) MOLECULAR BREEDING METHOD FOR WHEAT FUSARIUM HEAD BLIGHT-RESISTANCE

(71) Applicants: CROP RESEARCH INSTITUTE. SHANDONG ACADEMY OF AGRICULTURAL SCIENCES, Jinan City (CN); JIANGSU LIXIAHE REGIONAL AGRICULTURAL RESEARCH INSTITUTE, Yangzhou City (CN); SHANDONG ACADEMY OF AGRICULTURAL SCIENCES, Jinan City (CN)

(72) Inventors: JIANJUN LIU, JINAN CITY (CN); WENJING HU, YANGZHOU CITY (CN); HAOSHENG LI, JINAN CITY (CN); XUEYAN CHEN, HANZHONG CITY (CN); SHENGNAN ZHAI, JINAN CITY (CN); XINYOU CAO, JINAN CITY (CN); JIANMIN SONG, JINAN CITY (CN); JUN GUO, LINQU COUNTY (CN); CHENG LIU, CHENGDU (CN); AIFENG LIU, JINAN CITY (CN); DUNGONG CHENG, JINAN CITY (CN); XIUSHENG CHU, JINAN CITY (CN); FAJI LI, JINAN CITY (CN); RAN HAN, XIANYANG CITY (CN); YAN ZI, JINAN CITY (CN); XIAOLU WANG, JINAN CITY (CN)

(21) Appl. No.: 17/686,729

(22) Filed: Mar. 4, 2022

(30) Foreign Application Priority Data
Apr. 29, 2021 (CN) 2021104713722

(51) Int. Cl. A01H 1/04

(52) U.S. Cl. CPC A01H 1/045

(57) ABSTRACT

Disclosed is a molecular marker polymerization wheat breeding method for Fusarium head blight-resistance. The method uses an intermediate material for Fusarium head blight-resistance as a resistance source, and excellent varieties (strains) in a northern part of Huanghuai wheat production area as agronomic parents, and uses molecular marker-assisted selection and conventional breeding techniques. The molecular markers are a linked marker LJJT-1 of a Fusarium head blight-resistant gene Fhb1 and linked markers LJJ-2 and LJJ-3 of a Fusarium head blight-resistant gene Fhb2, so as to create a semi-winter Fusarium head blight-resistant breeding material with outstanding target traits and excellent comprehensive traits, which lays a material foundation for cultivating Fusarium head blight-resistant wheat varieties suitable for planting in the Huanghuai wheat production area.

### TECHNICAL FIELD

The disclosure belongs to the technical field of wheat breeding methods, and relates to a molecular breeding method for wheat Fusarium head blight-resistance.

### BACKGROUND

Fusarium head blight (FHB) is a serious ear disease caused by Fusarium graminearum Schwabe. Fusarium head blight has always been the most serious disease affecting wheat yield and quality in wheat production areas in the middle and lower reaches of the Yangtze River, spring wheat production areas in Northeast China, and wheat production areas in South China. In recent years, under the influence of climate warming, the promotion of dwarf varieties, the improvement of multiple cropping index and straw reuse in the field, the epidemic area of wheat FHB is expanding rapidly expanding from the middle and lower reaches of the Yangtze River to the Huanghuai winter wheat production area and even the northern winter wheat production area, and changing from an occasional disease to a frequent and major disease. In 2012, wheat FHB was a pandemic in China, and the affected area of wheat nationwide reached 10 million hectares. FHB not only affects the yield and quality of wheat, but also produces toxins such as vomitoxin (DON), which cause great hidden hazards to food safety. Breeding and promoting disease-resistant varieties is the most economical, effective, safe and reliable way to prevent and control FHB.

The wheat FHB-resistant gene/QTL has been officially named as FHB-resistant gene, and the molecular markers or functional markers that have been closely linked to FHB-resistant gene are Fhb1-Fhb7. Fhb1, Fhb2, Fhb4, Fhb5 are derived from common wheat, while Fhb3 and Fhb7 are from peripheral germplasm materials. Fhb1 and Fhb7 were cloned successively from 2019 to 2020. The Fhb1 gene has been recognized as the FHB-resistant gene with the most effective and most stable resistance so far. It is generally believed that this gene locus has a good anti-expansion effect, but some studies have shown that this locus has both anti-infection effect and anti-DON effect. Fhb2, derived from chromosome 6BS, is the second main gene showing resistance to expansion in common wheat after Fhb1 (Cuthbert P A, Somers D J, Brule-Babel A. Mapping of Fhb2 on chromo some 6BS: a gene controlling Fusarium head blight field resistance in bread wheat (Triticum aestivum L.) [J]. Theoretical&Applied Genetics, 2007, 114:429-437).

By cross-breeding among varieties, a number of resistant and moderately disease-resistant varieties that can be used in breeding and production have been created and cultivated in the wheat production areas of the middle and lower reaches of the Yangtze River. At present, most of the wheat varieties in the Huanghuai winter wheat production area, especially the Northern Huanghuai are highly susceptible to FHB, and there are lack of FHB-resistant varieties. With the development of molecular biology, the localization of disease-resistant genes and molecular marker technology have become increasingly perfect and mature, and molecular marker-assisted breeding technology has been widely used.

Since 2012, improving the resistance of new wheat varieties to FHB has become one of the main breeding goals in the Huanghuai winter wheat production area. The selection of resistant sources and the creation of resistant materials are the basis for selecting and promoting FHB-resistant varieties. The varieties and germplasm materials with good FHB-resistance in the middle and lower reaches of the Yangtze River can be planted in spring and have poor cold resistance, which are difficult to be used in FHB-resistant wheat breeding in the Northern Huanghuai, and germplasm materials for winter and semi-winter wheat FHB-resistant sources are scarce, making the direct utilization of FHB-resistant sources difficult. Therefore, the use of molecular marker-assisted selection combined with conventional breeding can provide technical support for the creation of semi-winter FHB-resistant breeding materials with outstanding target traits and excellent comprehensive traits in this wheat production area. At present, there is no example of using molecular marker-assisted selection to breed FHB-resistant varieties in this wheat production area.

### SUMMARY

The object of the disclosure is to overcome the defects existing in the prior art, and to provide a molecular marker polymerization breeding method for wheat FHB-resistance. The method uses an intermediate FHB-resistant material created by Nanjing Agricultural University as a resistance source, and an excellent variety (strain) in the northern part of the Huanghuai wheat production area as the agronomic parent, and uses molecular marker-assisted selection and conventional breeding techniques to create a semi-winter FHB-resistant breeding material with outstanding target traits and excellent comprehensive traits, which has laid a material foundation for cultivating FHB-resistant wheat varieties suitable for planting in Huanghuai wheat production area.

The disclosure provides a molecular breeding method for wheat FHB-resistance, including the following steps:

S1: carrying out hybridization by taking the intermediate material carrying a plurality of FHB-resistant genes as the female parent, and the large-area popularized variety with an average yield of more than 500 kilograms as the male parent, and obtaining the first-generation hybrid seed;

S2: taking the first-generation hybrid seed in S1 as the female parent, and the intermediate material with excellent agronomic properties of powdery mildew-resistant as the male parent to hybridize into a F₁ generation, and harvesting the seeds of the F₁ generation;

S3: planting the seeds of the F₁ generation (propagating the F₁ generation), mixed harvesting and threshing to produce a F₂ generation;

S4: the F₂ generation is propagated in the field, and first individual plants with excellent comprehensive agronomic traits are selected from the propagated F₂ generation, the linked marker LJJT-1 of the FHB-resistant gene Fhb1 and the linked markers LJJ-2 and LJJ-3 of Fhb2 are detected and screened from the first individual plants to select second individual plants that are all positive in molecular marker detection of the linked marker LJJT-1 and the linked markers LJJ-2 and LJJ-3, and the selected second individual plants are harvested and threshed to produce a F₃ generation;

S5: in each of the F₃ and a F₄ generation, propagating in the field in a manner of same strains in a line, selecting strains with consistent traits and phenotypes, excellent comprehensive disease resistance and comprehensive agronomic traits, detecting and screening the linked marker LJJ-1 of the FHB-resistant gene Fhb1 and the linked markers LJJ-2 and LJJ-3 of Fhb2 from the selected strains to select the second strains that are positive in molecular marker detection of the linked marker LJJT-1 and the linked markers LJJ-2 and LJJ-3, and mixing and threshing 3-5 homozygous-individual plants of the second strains; producing a F₅ generation, measuring the grains' yield, and screening out the strains with high yield;

S6: in the selected F₅ strains, inoculating the selected strains by adopting the single flower drip method, inoculating each strain with 30 ears; identifying the resistance of FHB, and harvesting the strains with the average yield per 666.7 m² of more than 500 kg, which has better FHB resistance than the medium resistance control group, and close to the disease-resistant control group.

In an embodiment, planting in S1 to S3 is carried out in a greenhouse.

In an embodiment, in S4, the excellent comprehensive agronomic traits mean that the selection is mainly based on cold tolerance, plant height, plant type, ear traits, grain color and powdery mildew resistance, etc., and a single plant with excellent comprehensive agronomic traits is selected.

In an embodiment, in the F₃-F₄ generation, the control variety Jimai 22 in the Huanghuai wheat production area is used as the reference for agronomic traits.

In an embodiment, in S6, the selected F₅ strains are planted in the field as an evaluation nursery according to the plot, and the control variety Jimai 22 in Huanghuai wheat production area is taken as the reference for agronomic traits, and the yield and quality are identified comprehensively, and one identification is repeated in a plot of six rows with plot length of 4 m and width of 1.5 m, mechanical sowing in drill and 150,000 basic seedlings/666.7 m². For the investigation of agronomic traits such as seedling habit, plant type, grain type and plant height, refer to the Technical Specification for Regional Tests of Crop Varieties—Wheat (NY/T 1301-2007).

In an embodiment, the intermediate material carrying the plurality of FHB-resistant genes is wheat NMAS020, which is an FHB-resisting intermediate material created by Nanjing Agricultural University using the near-isogenic line of the main FHB-resistant locus of Wangshuibai and PH691 for hybridization. The wheat NMAS020 contains four FHB-resistant genes, including Fhb1 (located on chromosome 3B), Fhb2 (6B), Fhb4 (4B) and Fhb5 (5A).

In an embodiment, the large-area popularized variety is Jimai 22, which is a wheat variety bred by Shandong Academy of Agricultural Sciences. Jimai 22 participated in the Shandong Provincial Regional Test and ranked first in both years, with an average yield of 536.81 kg per 666.7 m² and an extremely significant increase of 10.79% compared to the control; the average yield per 666.7 m² in the production test was 519.1 kg, an increase of 4.05% compared with the control; the average yield per 666.7 m² was 518.08 kg in the national Northern Huanghuai area test, a significant increase of 4.67% compared with the control group, and the average yield in the production test increased by 2.05% compared with the control group.

In an embodiment, the intermediate material with excellent agronomic properties of the powdery mildew-resistant is Shi H083-366, which is the intermediate material of Shijiazhuang Academy of Agricultural Sciences. The plant is relatively short, the plant type is compact and contains Pm21, and the plant is resistant to powdery mildew.

In an embodiment, the primer sequences of the linked marker LJJ-1 of the FHB-resistant gene Fhb1 are shown in SEQ ID NO: 1 and SEQ ID NO: 2; the primer sequences of the linked marker LJJ-2 of the FHB-resistant gene Fhb2 are as shown in SEQ ID NO: 3 and SEQ ID NO: 4; the primer sequences of the linked marker LJJ-3 of the FHB-resistant gene Fhb2 are as shown in SEQ ID NO: 5 and SEQ ID NO: 6.

PCR amplification is used to detect the corresponding linked marker LJ J-1 of the main FHB-resistant gene Fhb1. The PCR amplification is as follows: the PCR amplification system is 10 microliters (μL), including 1.0 μL of 30 nanograms per microliter (ng/uL) wheat genome DNA, 1.0 μL of 10×PCR buffer, 0.2 uL of 10 millimoles (mM) dNTP, 1.0 μL of 10 mM MgCl₂, 0.2 uL of 5 units (U) Taq polymerase, 0.4 uL of 5 micromoles (uM) upstream primer, 0.4 uL of 5 uM downstream primer and 5.8 uL of sterile deionized water; the PCR amplification procedure is as follows: pre-denaturation for 5 minutes (min) at 94 Celsius degrees (° C.); denaturation for 30 seconds (s) at 94° C., annealing for 30 s at 64° C., extension for 45 s at 72° C., 35 cycles; extension for 10 min at 72° C.; preservation at 4° C. LJJ-1 primers are used to determine the materials involved in this study together with their parents in 1% agarose electrophoresis solution. The target genotype is the same as NMAS020, which is the selected material.

The corresponding linked markers LJ2 and LJJ-3 of the main FHB-resistance gene Fhb2 are detected in PCR amplification way. The method of PCR amplification is as follows: the PCR amplification system is 10 μL, including 1.0 μL, of 30 ng/uL wheat genome DNA, 1.0 μL of 10×PCR buffer, 0.2 uL of 10 Mm dNTP, 1.0 μL, of 10 Mm MgCl₂, 0.2 uL of 5U Taq polymerase, 0.4 uL of 5 uM upstream primer, 0.4 uL of 5 uM downstream primer and 5.8 uL of sterile deionized water; the PCR amplification procedure is as follows: (1) pre-denaturation for 8 min at 94° C., (2) denaturation for 30 s at 94° C., (3) LJJ-2 primer annealed at 60° C. for 40 s; LJJ-3 primer annealed at 61° C. for 40 s, (4) extension for 30 s at 72° C., 36 cycles, (5) extension for 10 min at 72° C.; (6) preservation at 4° C. Electrophoresis with LJJ-2 and LJJ-3 primers in 8% non-denatured polyacrylamide gel electrophoresis solution for 1 hour and 40 min-2 hours and 30 min is carried out, the conditions are arc: bis=19:1 and 200 volts, and the materials involved in this research together with the parents are detected. The target genotype the same as NMAS020 is regarded as positive and is the selected material.

In an embodiment, the disease-resistant control group is Sumai No. 3, and the medium resistance control group is Zheng 9023.

In an embodiment, the fact that the FHB-resistant variety is better than that of the medium-resistance variety means that the average diseased spikelet number and the average diseased spikelet rate of the strain after 25 days of inoculation are less than that of the medium-resistance variety.

In an embodiment, the culture used in the single-flower drip method inoculation is F.g (Fusarium graminearum) 15-A DON type from Jiangsu Academy of Agricultural Sciences Institute of Food Quality Safety and Testing, which was donated by the Institute of Food Quality, Safety and Testing, Jiangsu Academy of Agricultural Sciences, and was propagated and provided by Chu Xiusheng, Institute of Crops, Shandong Academy of Agricultural Sciences, and was identified by artificial inoculation using the single-flower dripping method; a micropipette is used to take 20 μL of meristem suspension and inject it into the florets just bloomed on the middle spikelet of the wheat ear; 5 ears of each variety are inoculated, the wheat ear with a fresh-keeping bag for 3 days is inoculated, and the number of diseased spikelets and the total number of spikelets of each inoculated ear inoculation are calculated after 21 days, and the average number of diseased spikelets and the average diseased spikelet rate (%) are calculated as the evaluation index of FHB-resistance (Xue S L, Xu F, Tang M Z, et al. Predise mapping Fhb5, a major QTL conditioning resistance to Fusarium infection in bread wheat (Triticum aestivumL.) [J]. Theoretical&Applied Genetics, 2011, 123 (6): 1055T063).

Compared with the prior art, the application has the following technical effects:

1) the analysis of the application verifies that FHB resistance is different if varieties contain different disease-resistant genes and different disease-resistant gene combinations. Among the four types of strains containing only one disease-resistant gene, the average number of diseased spikelets of the strains containing Fhb1 and Fhb4 is significantly lower than that of the strains containing Fhb2 and Fhb5 types and the control Jimai 22, showing better FHB resistance; in the combination containing 2 disease-resistant genes, the average susceptible spikelet number and average susceptible spikelet rate of strains containing (Fhb1+Fhb2) are significantly lower than those of (Fhb1+Fhb4) strain. There is a significant difference in the performance when a variety contains a single disease-resistant gene or two of the same type of disease-resistant genes, which shows that the disease resistance effect of disease-resistant gene combinations is not predictable. The application utilizes molecular marker-assisted selection and conventional breeding technology, of which the molecular markers are the linked markers LJJ-1 and LJJ-2 of the FHB-resistant genes Fhb1 and Fhb2, and the specific combination could improve the efficiency of FHB-resistant variety breeding.

2) According to the application, the semi-winter FHB-resistant breeding materials Jimai 8681 and Jimai 8775 with outstanding target traits and excellent comprehensive traits are created, which lay a material foundation for cultivating FHB-resistant wheat varieties suitable for planting in Huanghuai wheat production area.

### BRIEF DESCRIPTION OF DRAWINGS

In order to more clearly illustrate the specific embodiments of the application or the technical solutions in the prior art, the following briefly introduces the accompanying drawings required in the description of the specific embodiments.

FIGS. 1A-1D show the FHB resistance performance and field appearance of wheat new varieties Jimai 8681 and Jimai 8775.

### DETAILED DESCRIPTION OF THE EMBODIMENTS

Embodiments of the technical scheme of the application will be described in detail below with reference to the accompanying drawings. The following examples are only used to more clearly illustrate the technical schemes of the application, and are therefore only used as examples, and cannot be used to limit the protection scope of the application. It should be noted that, unless otherwise specified, the technical terms or scientific terms used in this application should be the usual meanings understood by those skilled in the art to which the application belongs.

### Embodiment 1 Construction of Breeding Population of FHB Resistance and Breeding Method of Hybrid Variety

Hybrid F₁ is obtained by crossing with NMAS020 as the female parent and Jimai 22 as the male parent. Hybrid F₁ is used as the female parent, and Shi H083-366 containing the powdery mildew-resistant gene Pm21 is used as the male parent for multiple crossing. The main purpose of multiple crossing is to improve the powdery mildew resistance of hybrid variety. After multiple crossing, the pedigree method is used for selecting and breeding according to cold tolerance, plant height, plant type, ear traits, grain color and powdery mildew resistance. Molecular markers are used to track and detect FHB-resistant genes in individual plants and strains. For 53 strains with basically stable traits and excellent comprehensive traits, the single flower dripping method is used to identify FHB resistance, and Jimai 22 is the susceptible control.

The marker-specific primer sequences closely linked to FHB-resistant gene Fhb1, Fhb2 are shown in Table 1.

TABLE 1
Fhb1 and Fhb2 linked marker primer sequence information

| | | | | Annealing |
|---|---|---|---|---|
| | Linked | Primer | | temperature |
| Gene | marker | sequence (5′-3′) | Serial number | (°C.) |
| Fhb1 | LJJ-1-F | ATTCCTACTAGCCGCCTGGT | SEQ ID NO: 1 | 64 |
| | LJJ-l-R | ACTGGGGCAAGCAAACATTG | SEQ ID NO: 2 | |
| Fhb2 | LJJ-2-F | GGAGATTGACCGAGTGGAT | SEQ ID NO: 3 | 60 |
| | LJJ-2-R | CGTGAGAGCGGTTCTTTG | SEQ ID NO: 4 | |
| | LJJ-3-F | ATGCCTGCTTGCTCACTG | SEQ ID NO: 5 | 61 |
| | LJJ-3-R | TCCTATGCGTTCGGTTGG | SEQ ID NO: 6 | |

To detect the corresponding linked marker LJJ-1 of the main gene Fhb1 for FHB-resistance, the PCR amplification is as follows: the PCR amplification system is 10 μL, including 1.0 μL of 30 ng/uL wheat genome DNA, 1.0 μL of 10×PCR buffer, 0.2 uL of 10 Mm dNTP, 1.0 μL of 10 Mm MgCl₂, 0.2 uL of 5U Taq polymerase, 0.4 uL of 5 uM upstream primer, 0.4 uL of 5 uM downstream primer and 5.8 uL of sterile deionized water; the PCR amplification procedure is as follows: pre-denaturation for 5 min at 94° C.; denaturation for 30 s at 94° C., annealing for 30 s at 64° C., extension for 45 s at 72° C., 35 cycles; extension for 10 min at 72° C.; preservation at 4° C. LJJ-1 primers are used to detect the materials involved in this study together with their parents in 1% agarose electrophoresis solution. The target genotype is the same with NMAS020, which is the selected material.

The corresponding linked markers LJ2 and LJJ-3 of the main gene Fhb2 for FHB-resistance are detected by PCR amplification. The PCR amplification is as follows: the PCR amplification system is 10 μL, including 1.0 μL of 30 ng/uL wheat genome DNA, 1.0 μL of 10×PCR buffer, 0.2 uL of 10 Mm dNTP, 1.0 μL of 10 Mm MgCl₂, 0.2 uL of 5U Taq polymerase, 0.4 uL of 5 uM upstream primer, 0.4 uL of 5 uM downstream primer and 5.8 uL of sterile deionized water; the PCR amplification procedure is as follows: (1) pre-denaturation for 8 min at 94° C., (2) denaturation for 30 s at 94° C., (3) LJJ-2 primer annealed at 60° C. for 40 s; LJJ-3 primer annealed at 61° C. for 40 s, (4) extension for 30 s at 72° C., 36 cycles, (5) extension for 10 min at 72° C.; (6) preservation at 4° C. Electrophoresis with LJJ-2 and LJJ-3 primers in 8% non-denatured polyacrylamide gel electrophoresis solution is carried out, arc: bis=19:1, 200 volts electrophoresis for 1 hour 40 min-2 h 30 min, and detect the materials involved in this research together with the parents. The target genotype, which is the same as NMAS020, is regarded as positive and the selected material.

Tightly linked marker-specific primers and amplification of Fhb4 and Fhb5 refer to the article (Xue S L, LiG Q, Jia H Y, et al. Fine mapping Fhb4, a major QTL conditioning resistance to Fusarium infection in bread wheat (Triticum aestivum L.) [J]. Theoretical & Applied Genetics, 2010, 121 (1):147-156. Xue S L, Xu F, Tang M Z, et al. Predise mapping Fhb5, a major QTL conditioning resistance to Fusarium infection in bread wheat (Triticum aestivum L.) [J]. Theoretical & Applied Genetics, 2011, 123 (6):1055-1063.).

Fifty-three strains and the control Jimai 22 are planted in 15 fields of the Crop Institute of Shandong Academy of Agricultural Sciences, arranged in sequence, and one identification is repeated in a plot of six rows with plot length of 4 m and width of 1.5 m, mechanical sowing in drill and 150,000 basic seedlings/666.7 m². For the investigation of agronomic traits such as seedling habit, plant type, grain type and plant height, refer to the Technical Specification for Regional Tests of Crop Varieties—Wheat (NY/T 1301-2007).

The culture for the identification of Fusarium head blight is F.g (Fusarium graminearum) 15-A DON type, by Jiangsu Academy of Agricultural Sciences Institute of Food Quality Safety and Testing, which was donated by the Institute of Food Quality, Safety and Testing, Jiangsu Academy of Agricultural Sciences, and was propagated and provided by Chu Xiusheng, Institute of Crops, Shandong Academy of Agricultural Sciences, and was identified by artificial inoculation by single-flower drip method; using a micropipette to take 20 μL of meristem suspension and injecting it into the florets just bloomed on the middle spikelet of the wheat ear; inoculating 5 ears of each variety, inoculating the wheat ear with a fresh-keeping bag for 3 days, and investigating the number of diseased spikelets and the total number of spikelets of each inoculated ear 21 days after inoculation, and calculating the average number of diseased spikelets and the average diseased spikelet rate (%) as the evaluation index of Fusarium head blight-resistant. Breeding practice shows that although the FHB-resistant materials such as Sumai NO. 3 and Wangshuibai and their derived strains have good FHB-resistance, their agronomic traits, resistance to other diseases and high yield are not satisfactory. The gene linkage relationship often leads to the loss of the target gene for FHB-resistance during comprehensive trait selection. The multiple crossing breeding population for FHB-resistant of the present application finally retains 53 strains after continuous multi-generation field selection of excellent individual plants and strains for agronomic traits. According to molecular marker detection of FHB-resistance, 33 out of 56 strains contained 1-3 disease-resistant genes, accounting for 62.3%; there are 20 genes without any loci, accounting for 37.7% (Table 2). Although each generation of hybrids has been tested for molecular markers, and 15 gene combination types (including 4 gene combination types, Fhb1+Fhb2+Fhb4+Fhb5) appeared in the early generation, it may be due to the selection of comprehensive agronomic traits in the field, and only 7 types of gene combinations appeared.

TABLE 2
Number of strains with different genes and combinations

| | | | Average | |
|---|---|---|---|---|
| | | Average | diseased | |
| | | number of | spikelet | |
| | Number of | diseased | rate | Amplitude of |
| Gene | strains | spikelets | (%) | variation |
| None | 20 | 16.0 | 85.2 | 41.9-100.0 |
| Fhh1 | 3 | 9.6 | 49.0 | 14.0-91.2 |
| Fhb2 | 2 | 14.4 | 83.5 | 67.0-100.0 |
| Fhh4 | 8 | 9.9 | 54.8 | 34.9-91.8 |
| Fhb5 | 1 | 15.2 | 75.8 | — |
| Fhbl + Fhb2 | 3 | 2.1 | 10.2 | 5.1-16.5 |
| Fhbl + Fhb4 | 5 | 6.2 | 33.8 | 12.0-100.0 |
| Fhb1 + Fhb2 + | 11 | 1.6 | 9.9 | 4.8-26.2 |
| Fhb5 | | | | |

In general, the average number of diseased spikelets and the average diseased spikelet rate with different numbers of FHB-resistant genes are significantly different. The average number of diseased spikelets and the average rate of diseased spikelets with three FHB-resistant genes are significantly lower than those with two FHB-resistant genes, those with two FHB-resistant genes are significantly lower than those with one FHB-resistant gene, and those with one FHB-resistant gene are significantly lower than the strain without any FHB-resistant genes and control variety Jimai 22 (Table 3). According to the grading standard and evaluation standard for the severity of FHB (agricultural industry standard), the variety containing 1 disease-resistant main gene can reach the level of moderate susceptibility, the variety containing 2 genes can reach the level of moderate resistance and above, and the variety containing 3 disease-resistant genes can reach the level of high resistance. Varieties (strains) without any main gene (including the control Jimai 22) show high susceptibility to FHB. Therefore, the combination of FHB-resistant genes (Fhb1+Fhb2) is selected as the molecular selection marker in FHB-resistant breeding.

TABLE 3
Average performance of FHB in strains with different
numbers of disease-resistant genes

| | | Average diseased |
|---|---|---|
| Number of disease- | Average number of | spikelet rate |
| resistant genes | diseased spikelets | (%) |
| 0 | 16.0 | 85.2 |
| 1 | 12.3 | 65.8 |
| 2 | 5.8 | 32.0 |
| 3 | 1.6 | 9.9 |
| Control (Jimai 22) | 15.3 | 84.4 |

### Embodiment 2

S1: in April of the first year, using the intermediate material NMA S020 carrying multiple FHB-resistant genes as the female parent, and the large-area popularized variety Jimai 22 with an average yield of more than 500 kg/666.7 m² as the male parent in the greenhouse to hybridize into hybrid F₁ and obtain hybrid F₁ seeds;

S2: in April of the second year, continuously using hybrid F₁ as the female parent in the greenhouse, and the intermediate material Shi H083-366 with excellent agronomic properties containing powdery mildew-resistant gene Pm21 as the male parent to hybridize into F₁ generation in the greenhouse, and harvesting the seeds.

S3: in April of the third year, continuously propagating F₁ generation in the greenhouse, mixing, harvesting and threshing to produce F₂ generation.

S4: in October of the fourth year, performing propagation of F₂ generation in the field, selecting individual plants with excellent comprehensive agronomic traits mainly according to cold tolerance, plant height, plant type, ear traits, grain color and powdery mildew-resistance, etc., detecting and screening individual plants, selecting individual plants which are positive in molecular marker detection, and harvesting and threshing the selected single plants to produce F₃ generation;

Wherein the marker-specific primer sequences closely linked to the FHB-resistant gene Fhb1, Fhb2 are shown in Table 3.

S5: in October of the fifth year, propagating F₃ generation in the field in a manner of same strains in a line, and setting the reference variety Jimai 22 in the Huanghuai wheat production area as the reference for agronomic traits; selecting strains with consistent traits and phenotypes and with good comprehensive disease-resistant and agronomic traits, detecting and screening individual plants with the linked marker LJJT-1 of the FHB-resistant gene Fhb1 and the linked markers LJJ-2 and LJJ-3 of Fhb2 according to the aforementioned amplification steps, selecting individual plants that are positive in molecular marker detection, and harvesting the 3-5 homozygous individual plants of each of the selected strains, mixing and threshing to produce F₄ generation, measuring the grain yield, and screening the strains with high yields.

S6: propagating the selected F₄ strain in the field in three rows in October of the sixth year, and setting the Huanghuai wheat production area control variety Jimai 22 as a reference for agronomic traits; selecting strains with consistent traits and phenotypes and with good comprehensive disease-resistant and agronomic traits; detecting and screening individual plants with the linked marker LJJT-1 of the FHB-resistant gene Fhb1 and the linked markers LJJ-2 and LJJ-3 of Fhb2 according to the aforementioned amplification steps, selecting individual plants that are positive in molecular marker detection, harvesting, mixing and threshing the 3-5 homozygous individual plants of each of the selected strains to produce F₅ generation, measuring the spot yield, and screening 3 strains with the highest yield.

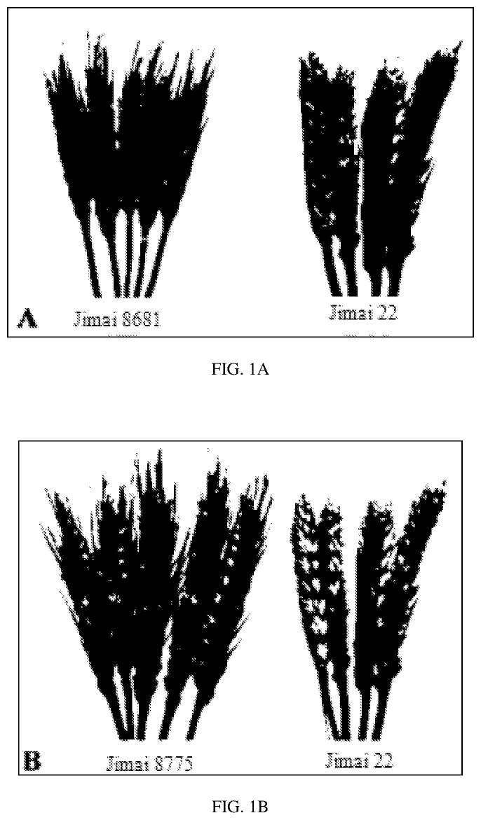

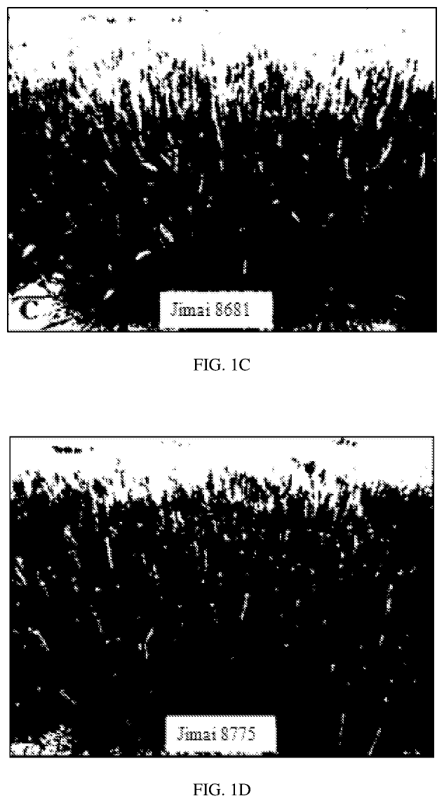

S7: in October of the seventh year, planting the selected F₅ strains in the Datiancheng evaluation nursery according to the plot, setting the reference variety Jimai 22 in the Huanghuai wheat production area as the reference for agronomic traits, identifying the yield and quality comprehensively, and repeating once in a plot of six rows with plot length of 4 m and width of 1.5 m, mechanical sowing in drill and 150,000 basic seedlings/666.7 m²; For the investigation of agronomic traits such as seedling habit, plant type, grain type and plant height, referring to the Technical Specification for Regional Tests of Crop Varieties—Wheat (NY/T 1301-2007). Inoculating selected strains by single-flower drip method. Inoculating each strain with 30 ears to identify the resistance of FHB. Jimai 22 is the susceptible control, Sumai NO. 3 is disease-resistant control, and Zheng 9023 is the medium resistance control; obtaining the strains Jimai 8681 and Jimai 8775 (Table 1, FIG. 1A and FIG. 1B), which have better resistance to FHB than Zheng 9023 and are close to Sumai No. 3 with an average yield of more than 500 kg.

The culture for identifying FHB is F.g (Fusarium graminearum) 15-A DON type, which was donated by the Institute of Food Quality, Safety and Testing, Jiangsu Academy of Agricultural Sciences, and was propagated and provided by Chu Xiusheng, Institute of Crops, Shandong Academy of Agricultural Sciences, and was identified by artificial inoculation with the single-flower drip method; a micropipette is used to take 20 μL of meristem suspension and inject it into the florets just blooming on the middle spikelet of the wheat ear; inoculating 5 ears of each variety, inoculating the wheat ear with a fresh-keeping bag for 3 days, and investigating the number of diseased spikelets and the total number of spikelets of each inoculated ear 21 days after inoculation, and calculating the average number of diseased spikelets and the average diseased spikelet rate (%) as the evaluation index of FHB-resistance (Xue S L, Xu F, Tang M Z, et al. Predise mapping Fhb5, a major QTL conditioning resistance to Fusarium infection in bread wheat (Triticum aestivum L.) [J]. Theoretical&Applied Genetics, 2011, 123 (6):1055-1063).

TABLE 4
Detection results of FHB resistance of Jimai 8681 and Jimai 8775

| | | | Average | Average |
|---|---|---|---|---|
| | | | number of | diseased |
| | | | diseased | spikelet |
| Name of variety | F | Fhb | spikelets | rate |
| Jimai 8681 | + | + | 2.2 | 10.5 |
| Jimai 8775 | + | + | 2.0 | 9.5 |
| Jimai 22 (susceptible control) | − | − | 12.5 | 67.9 |
| Zheng 9023 (medium resistance control) | − | − | 5.2 | 25.8 |
| Sumai No. 3 (disease-resistant control) | + | + | 1.0 | 5.0 |

Jimai 8681 has better comprehensive agronomic traits (FIG. 1C, Table 5). The seedlings of this strain are semi-prostrate, with narrow leaves, light seedling colors, and good cold resistance (grade 2); the plant type is compact, with a height of 78 cm; compared with the control Jimai 22, the heading stage of Jimai 8681 is about 5 days earlier, and the maturity stage is 3 days earlier, so the variety is mid-early maturing; the variety has oval ears, long awns, white husks, white grain, oval grains, and the thousand-grain weight of 45 g. The variety contains powdery mildew-resistant gene Pm21. The yield per plot is 530.1 kg/666.7 m², an increase of 3.29% compared with the control Jimai 22.

Jimai 8775 has better comprehensive agronomic traits (FIG. 1D, Table 5). The seedlings of this strain are semi-erect with narrow leaves, dark colors, and good cold resistance (grade 2); the seedlings have a height of 81 cm; the heading stage is 5 days earlier than control Jimai 22, and the maturity stage is 3 days earlier, so the variety is a mid-early maturing variety; the variety has oblong ears long awns, white husks, white and oval grains, the thousand-grain weight of 42 g. The yield per plot is 520.9 kg/666.7 m², an increase of 0.85% compared with the control Jimai 22.

TABLE 5
Comprehensive agronomic traits of Jimai 8681 and Jimai 8775

| | | height/ | Type, cold | Thousand-grain | Fusarium | |
|---|---|---|---|---|---|---|
| Variety | Growing period | cm | tolerance | weight/g | head blight | Yield |
| Jimai | Compared with the control Jimai 22, the | 78 | Compact, | 45 | Medium | 530.1 kg/666.7 m², |
| 8681 | heading stage is about 5 days earlier, and | | good cold | | resistance | an increase of 3.29% |
| | the maturity stage is 3 days earlier, and | | resistance | | | compared with the |
| | is a mid-early maturing variety | | | | | control Jimai 22. |
| Jimai | Heading stage is 5 days earlier than control | 81 | Compact, | 42 | Medium | 520.9 kg/666.7 m², |
| 8775 | Jimai 22, and the maturity stage is 3 days | | good cold | | resistance | an increase of 0.85% |
| | earlier, and is a mid-early maturing variety | | resistance | | | compared with the |
| | | | | | | control Jimai 22. |
| Jimai | mid-lately maturing variety; the maturity | 75 | Compact, | 40.4 | Susceptible | 518.1 kg/666.7 m² |
| 22 | stage is 1 day longer than that of control | | poor cold | | | |
| | Shi 4185 | | resistance | | | |

To sum up, according to the molecular marker polymerization breeding method for FHB-resistance in wheat of the present application, the linked marker LJJT-1 of the molecular markers FHB-resistant gene Fhb1 and the linked markers LJJ-2 and LJJ-3 of Fhb2 are used in the breeding generation using molecular marker-assisted selection and conventional breeding techniques, so as to create a semi-winter FHB-resistant breeding materials Jimai 8681 and Jimai 8775 with outstanding target traits and excellent comprehensive traits, which has laid a material foundation for cultivating FHB-resistant wheat varieties suitable for planting in Huanghuai wheat production area.

Unless specifically stated otherwise, the numerical values set forth in these examples do not limit the scope of the disclosure. In all examples shown and described herein, unless stated otherwise, any specific value should be construed as merely exemplary and not as limiting, as other examples of exemplary embodiments may have different values.

1-5. (canceled)

6. A molecular marker-assisted breeding method for wheat Fusarium head blight (FHB)-resistance, and the method comprising:
S1: crossing a first wheat plant comprising a plurality of FHB-resistance genes with a second wheat plant with an average yield of more than 500 kilograms per 666.7 m² to obtain a first-generation hybrid seed; wherein the plurality of FHB-resistance genes comprises: a FHB-resistance gene Fhb1 and a FHB-resistance gene Fhb2;
S2: crossing the first-generation hybrid seed with a third wheat plant comprising a powdery mildew resistance gene to obtain a F₁ generation;
S3: propagating the F₁ generation, and harvesting in a mixed way and threshing to obtain a F₂ generation;
S4: propagating the F₂ generation, and selecting first individual plants with cold tolerance, powdery mildew resistance, and plant height, plant type, ear shape and grain color meeting target requirements; detecting and screening second individual plants being positive in a marker LJJ-1 of the FHB-resistance gene Fhb1 and markers LJJ-2 and LJJ-3 of the FHB-resistance gene Fhb2 in molecular marker detection from the first individual plants, and harvesting and threshing the second individual plants to obtain a F₃ generation;
S5: propagating the F₃ generation in a field in a manner of same strains in a line, selecting first strains with same traits and phenotypes as a target wheat plant and with powdery mildew resistance, detecting and screening second strains being positive in the marker LJJ-1 of the FHB-resistance gene Fhb1 and the markers LJJ-2 and LJJ-3 of the FHB-resistance gene Fhb2 in molecular marker detection from the first strains, and mixing and threshing 3-5 homozygous-individual plants of each of the second strains to obtain a F₄ generation, measuring grain yields of the F₄ generation and selecting initial target strains with highest grain yield from the F₄ generation;
S6: propagating the initial target strains of the F₄ generation in a field in a manner of same strains in a line, selecting third strains with same traits and phenotypes as the target wheat plant and with powdery mildew resistance, detecting and screening fourth strains being positive in the marker LJJ-1 of the FHB-resistance gene Fhb1 and the markers LJJ-2 and LJJ-3 of the FHB-resistance gene Fhb2 in molecular marker detection from the third strains, and mixing and threshing 3-5 homozygous-individual plants of each of the fourth strains to obtain a F₅ generation, and measuring yields of the F₅ generation and selecting top three strains with highest yield from the F₅ generation;
S7: performing inoculation on the top three strains of the F₅ generation by using a single flower dripping method to obtain strains after the inoculation, and each of the top three strains being inoculated with 30 ears; measuring FHB resistance of the strains after the inoculation to obtain target strains with better FHB resistance than a target control group; wherein an average yield of each of the target strains is more than 500 kilograms (kg) per 666.7 m²;
wherein primer sequences of the marker LJJ-1 of the FHB-resistance gene Fhb1 are shown in SEQ ID NO: 1 and SEQ ID NO: 2; primer sequences of the marker LJJ-2 of the FHB-resistance gene Fhb2 are shown in SEQ ID NO: 3 and SEQ ID NO: 4; and primer sequences of the marker LJJ-3 of the FHB-resistance gene Fhb2 are shown in SEQ ID NO: 5 and SEQ ID NO: 6.

7. A molecular marker-assisted breeding method for wheat Fusarium head blight (FHB)-resistance, and the method comprising:
S1: crossing a NMAS020 wheat plant comprising a plurality of FHB-resistance genes with a Jimai 22 wheat plant with an average yield of more than 500 kilograms per 666.7 m² to obtain a first-generation hybrid seed; wherein the plurality of FHB-resistance genes comprises: a FHB-resistance gene Fhb1 being detectable by a marker LJJ-1 and a FHB-resistance gene Fhb2 being detectable by markers LJJ-2 and LJJ-3;
S2: crossing the first-generation hybrid seed with a Shi H083-366 wheat plant comprising a powdery mildew resistance gene Pm21 to obtain a F₁ generation;
S3: propagating the F₁ generation, and harvesting in a mixed way and threshing to obtain a F₂ generation;
S4: propagating the F₂ generation, selecting first individual plants with cold tolerance, powdery mildew resistance, and plant height, plant type, ear shape and grain color meeting target requirements, detecting and screening second individual plants being positive in the marker LJJ-1 of the FHB-resistance gene Fhb1 and the markers LJJ-2 and LJJ-3 of the FHB-resistance gene Fhb2 in molecular marker detection from the first individual plants, and harvesting and threshing the second individual plants to obtain a F₃ generation;
S5: propagating the F₃ generation, selecting first strains with same traits and phenotypes as Jimai 22 and with powdery mildew resistance, detecting and screening second strains being positive in the marker LJJ-1 of the FHB-resistance gene Fhb1 and the markers LJJ-2 and LJJ-3 of the FHB-resistance gene Fhb2 in molecular marker detection from the first strains, and mixing and threshing 3-5 homozygous-individual plants of each of the second strains to obtain a F₄ generation, measuring grain yields of the F₄ generation and selecting initial target strains with highest grain yield from the F₄ generation;
S6: propagating the initial target strains, selecting third strains with same traits and phenotypes as the Jimai 22 and with powdery mildew resistance, detecting and screening fourth strains being positive in the marker LJJ-1 of the FHB-resistance gene Fhb1 and the markers LJJ-2 and LJJ-3 of the FHB-resistance gene Fhb2 in molecular marker detection from the third strains, and mixing and threshing 3-5 homozygous-individual plants of each of the fourth strains to obtain a F₅ generation, and measuring yields of the F₅ generation and selecting top three strains with highest yield from the F₅ generation;
S7: performing inoculation on the top three strains of the F₅ generation by using a single flower dripping method to obtain strains after the inoculation; measuring FHB resistance of the strains after the inoculation to obtain target strains with better FHB resistance than Zheng 9023; wherein an average yield of each of the target strains is more than 500 kilograms (kg) per 666.7 m²;
wherein primer sequences of the linked marker LJJ-1 of the FHB-resistance gene Fhb1 are shown in SEQ ID NO: 1 and SEQ ID NO: 2; primer sequences of the linked marker LJJ-2 of the FHB-resistance gene Fhb2 are shown in SEQ ID NO: 3 and SEQ ID NO: 4; and primer sequences of the linked marker LJJ-3 of the FHB-resistance gene Fhb2 are shown in SEQ ID NO: 5 and SEQ ID NO: 6.


SEQUENCE LISTING (6 sequences)

SEQ ID NO: 1 - DNA, 20 bp, artificial sequence
  primer sequence of the linked marker LJJ-1 of the Fhb1
attcctactagccgcctggt

SEQ ID NO: 2 - DNA, 20 bp, artificial sequence
  primer sequence of the linked marker LJJ-1 of the Fhb1
actggggcaagcaaacattg

SEQ ID NO: 3 - DNA, 19 bp, artificial sequence
  primer sequence of the linked marker LJJ-2 of the Fhb2
ggagattgaccgagtggat

SEQ ID NO: 4 - DNA, 18 bp, artificial sequence
  primer sequence of the linked marker LJJ-2 of the Fhb2
cgtgagagcggttctttg

SEQ ID NO: 5 - DNA, 18 bp, artificial sequence
  primer sequence of the linked marker LJJ-3 of the Fhb2
atgcctgcttgctcactg

SEQ ID NO: 6 - DNA, 18 bp, artificial sequence
  primer sequence of the linked marker LJJ-3 of the Fhb2
tcctatgcgttcggttgg